(19) United States
(12) Patent Application Publication
(10) Pub. No.: US 2003/0221203 A1
(43) Pub. Date: Nov. 27, 2003

(54) High efficiency regulatable gene expression system

(76) Inventors: Agha-Mohammadi Siamak, Pittsburgh, PA (US); Michael T Lotze, Pittsburgh, PA (US)

Correspondence Address: KIRKPATRICK & LOCKHART LLP, 535 SMITHFIELD STREET, PITTSBURGH, PA, 15222, US

(21) Appl. No.: 10/148,521

(22) PCT Filed: Oct. 3, 2001
(86) PCT No.: PCT/US01/31138

(51) Int. Cl. A01K 67/00; A01H 1/00; C12N 15/82; C07H 21/04; C12N 15/85

(52) U.S. Cl. 800/8; 800/278; 536/23.2; 435/455; 435/468

(57) ABSTRACT

Highly efficient regulated promoters (RPs) and transactivators are provided. The RPs contain 2-8 transactivator binding domains positioned in an optimized manner with respect to each other. In a tetracycline-regulatable system, the RP displays 5- to 10-fold enhanced regulation efficiency by maintaining high maximal expression while offering 5- to 10-fold reduced basal leakiness. In transient studies, these novel promoters display over 900-fold gene regulation at 1:1 ratio of transactivator to reporter plasmid. Furthermore, these promoters preserve their regulator efficienty in the context of a single positive feedback regulatory vector that presents ease of delivery of the system for many uses, including for use in vivo and in gene therapy. Finally, humanized transactivators are provided, for example a tetracycline transactivator utilizing the human transactivational domain of NF-κp65 protein fused to tetracycline repressor (terR) is provided that functions as efficiently as the tetracycline-regulated transactivator (tTA) and should reduce the potential immunogenicity of the original tTA.

### CROSS-REFERENCE TO RELATED APPLICATIONS

This application claims priority to U.S. Provisional Patent Application Serial No. 60/237,633, filed Oct. 3, 2000.

### STATEMENT REGARDING FEDERALLY SPONSORED RESEARCH OR DEVELOPMENT

This invention was made with governmental support under National Institutes of Health Biologic Therapy Research Career Development Grant No. K12CA76906. The government has certain rights in this invention.

### BACKGROUND

1. Field of the Invention

Provided is a high efficiency regulatable gene expression system including a promoter and a humanized transactivator. Also provided are methods for inducing expression of a nucleic acid using the regulatable gene expression system.

2. Description of the Related Art

Certain regulatable systems, such as tetracycline-regulatable systems (TRS), offer powerful tools for regulating gene expression. For effective in vivo gene regulation, these systems often suffer from inconsistent delivery and efficiency as double vectors, low efficiency as a single vector, high basal leakiness, and potential immunogenicity.

Gene expression systems for in vitro and in vivo work, especially mammalian expression systems, require 5 efficient delivery of genes to targeted cells as well as regulatable gene expression. For instance, most of the current gene delivery systems rely on strong viral promoters for in vivo expression of therapeutic genes. These promoters exhibit constitutive expression of proteins which can lead to down-regulation of effector systems and/or cellular toxicity. Recent studies on in vivo gene expression and gene therapy approaches have demonstrated the need for gene delivery vectors that can efficiently introduce and control expression of foreign genes in a dose-dependent manner. For many in vitro and in vivo applications, a dose-dependent expression system is desirable. Specifically, for correcting certain disorders in vivo, only a specific range or dose of a therapeutic protein will achieve a successful outcome (Dranoff, G., Jaffee, E., Lazenby, A., Golumbek, P., Levitsky, H., Brose, K., Jackson, V., Hamada, H., Pardoll, D. and Mulligan, R. C. (1993). Vaccination with irradiated tumor cells engineered to secrete murine granulocyte-macrophage colony-stimulating factor stimulates potent, specific, and long lasting anti-tumor immunity. Proc. Natl. Acad. Sci. USA. 90:3539-3543;Schmidt, W., Schweighoffer, T., Herbst, E., Maass, G., Berger, M., Schilcher, F., Schaffner, G., and Birnstiel, M. L. (1995). Cancer vaccines: The Interleukin 2 dosage effect. Proc. Natl. Acad. Sci. USA. 92:4711-4714; Naffakh, N. and Danos, O. (1996). Gene transfer for erythropoiesis enhancement. Mol. Medicine today. 2(8):343-348; each of which is incorporated herein by reference in its entirety). Hence in clinical practice, it will be beneficial to regulate gene expression in order to maintain protein concentrations within the therapeutic window and to optimize efficacy in response to the evolving nature of the disease.

Several chimeric regulatable systems incorporating various prokaryotic and eukaryotic elements have been devised to overcome the poor efficiency and generalized pleiotropic effects of earlier regulatable promoters. These systems employ drugs or hormones (or their analogs) as their inducing agents and utilize modular transcriptional transactivators composed of natural or mutant drug/hormone ligand binding domains, intrinsic or extrinsic DNA binding domains, and transcriptional activation domains and include, without limitation, the tetracycline-regulatable system, the progesterone-regulatable system, the ecdysone-regulatable system and the rapamycin-regulatable system (see, Agha-Mohammadi, S., and Lotze, M. T. (2000). Regulatable systems: applications in gene therapy and replicating viruses. J. Clin. Invest. 105:1177-1183, incorporated herein by reference). The tetracycline-regulatable system (TRS) (Gossen, M., and Bujard, H. (1992). Tight control of gene expression in mammalian cells by tetracycline-regulated promoters. Proc. Natl. Acad. Scd. USA. 89:5547-5551 and U.S. Pat. No. 6,136,954, which is incorporated herein by reference in its entirety as broadly disclosing and defining first generation tetracycline-regulatable systems) has proved to be an extremely popular tool, with more than 400 citations, for controlling gene expression both in vitro and in vivo (see, for example, Kistner, A., Gossen, M., Zimmerman, F., Jerecic, J., Ullmer, C., Lübbert, H. and Bujard, H. (1996) Proc. Natl. Acad. Sci. USA 93:10933-10938). Tetracycline-regulatable Vectors and systems as described above are commercially available from BD Biosciences Clontech (“Clontech”) under the trade names TET-OFF™ (tetracycline turns off gene expression) and TET-ON™ (tetracycline turns on gene expression).

To date, hundreds of researchers have used tetracycline-responsive promoters in a variety of applications, including: cell and gene therapies; veterinary uses; yeast expression systems; gene function studies; conditional immortalization of primary cells of therapeutic relevance; biocatalysis, fermentation and bioprocessing of therapeutic or other molecules; transgenic plants and animals; high throughput screening applications; functional genomics and target validation; and in plant biosciences, including establishment of disease and pest resistance.

The broadly available first-generation tetracycline-regulatable gene expression system (TRS) uses a conditionally active chimeric tTA (tetracycline transactivator) which was created by fusing the C-terminal 127 amino acids of herpes simplex virus VP16 to the carboxyl terminus of the tetR. In the absence of tetracycline, the tetR moiety of tTA binds with high affinity and specificity to a regulatory region comprising 7 repeats of a B2 tetO sequence built upstream of a minimal human CMV promoter to form tetracycline-regulated promoter (tRP). In this tRP, the palindromic centers of each adjacent pair of tetO sequences is 41 bases apart, or about four turns of the DNA helix. At this position, the VP16 moiety of tTA strongly induces transactivation of the distal target gene by promoting the assembly of a transcriptional initiation complex. Binding of tetracycline to tTA leads to a conformational change in tetR accompanied with loss of tetR affinity for tetOs (Gossen and Bujard, 1992).

Compared to the other existing chimeric systems, the TRS offers several advantages that make it particularly suitable for mammalian gene regulation (Agha-Mohammadi and Lotze, 2000). In this regard, the most important feature of TRS is its reliance on the tetracycline family of antibiotics as inducing agents. The well documented pharmacokinetics and pharmacodynamics of these drugs enable their use in an optimized fashion in human subjects. Furthermore, these agents cause little adverse effects on mammalian cells at the concentrations utilized for TRS modulation. Also, the system displays unique kinetics of regulation in response to different clinically used tetracycline analogs.

Using oxytetracycline, the system displays rapid switch-off and switch-on kinetics within 72 hours (Agha-Mohammadi, S., Alvarez-Vallina, L., Ashworth, L. J., and Hawkins, R. E. (1997). Delay in resumption of the activity of tetracycline-regulatable promoter following removal of tetracycline analogues. Gene Ther. 4,993-997, incorporated herein by reference in its entirety). Apart from these benefits, the system can be delivered conveniently as a single vector (Agha-Mohammadi, S., and Hawkins, R. E. (1998). Efficient transgene regulation from a single tetracycline-controlled positive feedback regulatory system. Gene Ther. 5(1):76-84, incorporated herein by reference in its entirety) or engineered to function in a down- or up-regulatory manner (Gossen, M., Freundlieb, S., Bender, G., Muller, G., Hillen, W., and Bujard, H. (1995). Transcriptional activation of tetracyclines in mammalian cells. Science 268:1766-1769; Deuschle, U., Meyer, W. K. H, and Thiesen, H. J. (1995). Tetracycline-reversible silencing of eukaryotic promoters. Mol. Cell. Biol. 15:1907-1914; Kim, H. J., Gatz, C., Hillen, W., and Jones, T. R. (1995). Tetracycline repressor-regulated gene repression in recombinant human cytomegalovirus. J. Virol. 69:2565-2573; each of which is incorporated herein by reference in its entirety).

With realization of the potential applications of TRS in gene therapy, its limitations have become more apparent. The system is often difficult and cumbersome to set-up. To regulate the expression of a desired gene using TRS, the two components of this system have to be co-expressed simultaneously in the same target cell. Also, the expression of the transactivator has to be high and the leakiness of the reporter gene has to be low. The result is frequently less than ideal, with most reports often managing a 10- to 100-fold regulation at best.

To simplify delivery, several single vectors have been described, but none of these have attained significantly high efficiency for in vivo applications (Agha-Mohammadi. S. and Hawkins R E. (1998)). In this article, several self-contained vectors were investigated carrying both the tetracycline-controlled transactivator (tTA) and a potentially therapeutic gene in transient studies. An enhancer-less positive feedback regulatory vector (pSiaIV) transcribing both tTA and mGM-CSF from a modified tTA-responsive bi-directional promoter demonstrated over 200-fold gene regulation in HeLa cells. That was comparable to the degree of regulation obtained on co-transfection of vectors expressing tTA and tTA-responsive mGM-CSF. The maximal transcriptional activity of pSiaIV was comparable to that of CMV IE promoter and its basal activity as low as the leakiness of the tetracycline-responsive promoter (tRP) in several commonly used cell lines, resulting in 47- to 328-fold regulation. Furthermore, pSiaIV also showed efficient regulation in stable cells.

Another disadvantage of the system is its high leakiness from the switched-off TRS. This not only reduces the efficiency of regulation but also prevents the use of the system for cytotoxic gene control. Finally, as a chimeric regulatable system, the TRS may have potential immunogenicity even though no anti-tTA antibody has been detected within a 6 month period (Bohl, D., Naffakh, N., and Heard, J. M. (1997). Long-term control of erythropoietin secretion by doxycycline in mice transplanted with engineered primary myoblasts. Nat. Med. 3:299-305, incorporated herein by reference in its entirety).

### SUMMARY

A gene expression system is thus provided that is suitable not only for use in vitro as a dosage-dependent regulatable gene expression system, but for use ex vivo, in vivo and in gene therapies. The expression system includes a control region including two or more transactivator DNA binding domains that are spaced such that when bound by a transactivators, the transactivators are substantially rotationally aligned about the DNA helix. In use, this control region is operably linked to a promoter to form a regulatable promoter (RP). The RP may be attached to a polylinker or recombination sequence which is also attached to a polyA signal opposite the RP, thereby producing an expression cassette into which a coding sequence may be inserted for regulatable expression of the coding sequence. The transactivators binding domains, RP or expression cassette may be inserted into a vector for either propagation of the vector or for gene expression in a cell.

This system displays high regulation efficiency. In the case of a tetracycline-regulatable system, including a tRP having two or more tetO sequences operably linked to a promoter, this efficiency is seen in both up-regulatory (as with a TET-ON™ system) or down-regulatory (as with a TET-OFF™ system) systems, as well as single or double plasmids. When used in combination with a humanized transactivator, such as the tetR-NF-κB p65 transactivator described below, this system is potentially less immunogenic.

One specific embodiment employs 23-29, 33-39 or 43-50, preferably about 26, 36 or 46 base pairs or about 2½, 3½ or 4½ helical turns between the central bases of each adjacent pair of 8 tetO sequences and, together with a minimal CMV promoter, forms a second generation tRP. This second generation tRP displays maximal expression 5- to 10-fold higher than the original tRP in the context of both up-regulatory and down-regulatory systems. By altering the distance between adjacent tetO sequences, the efficiency of the new tRP is increased by 5- to 10-fold from two orders of magnitude of regulation to three orders of magnitude.

The regulatable promoter systems described herein are completely interchangeable with the first generation promoter systems that are commercially available. As such, they are applicable in a large variety of in vitro, ex vivo and in vivo applications as described in the literature for the first generation systems, including, without limitation: cell and gene therapies; veterinary uses; yeast expression systems; gene function studies; conditional immortalization of primary cells of therapeutic relevance; biocatalysis, fermentation and bioprocessing of therapeutic or other molecules; transgenic plants and animals; high throughput screening applications; functional genomics and target validation; and in plant biosciences, including establishment of disease and pest resistance.

Also provided is a method for modulating expression from a regulatable promoter including two or more transactivator binding domains. The method includes the step of modifying the distance between the two or more domains to achieve a desired degree of regulation efficiency, that is to vary the maximal and basal transcription rates.

Lastly, a cell and transgenic plants or animals are provided including a gene controlled by the above-described RP.

### BRIEF DESCRIPTION OF THE DRAWINGS

FIG. 1 shows the sequences of the 2tetO inserts (SEQ ID NOS: 1-8). The tetO sequence is shown in bold with the central base, G, in italic. The random bases intervening between the 2 consecutive tetO sites is shown in normal font. The sequence intervening between the 2 central bases is underlined. Bps states for base pairs.

FIG. 2 is a graph showing the results of Example 3. The fold regulation of the inducement is indicated above the bar portions of the graph ((□)=Dox+; (▪)=Dox−)

### DETAILED DESCRIPTION

With recent advances in molecular biology and gene therapy, regulatable promoters have been much desired for modifying the timing and the dose of a gene product, both in vitro and in vivo. Recently, 4 chimeric regulatable systems have been constructed to overcome limitations of earlier regulatory promoters (Agha-Mohammadi and Lotze, 2000). Among these, the tetracycline regulatable system has proved to be an extremely valuable tool for controlling gene expression with several advantages relative to the other chimeric regulatable systems. However, even though the system has found much favor, it still suffers from difficulties in set-up, high basal leakiness, unpredictable delivery and efficiency, and potential immunogenicity.

With regard to the TRS, achieving regulated gene expression is still a cumbersome procedure, requiring several months to establish a stable cell line. In addition, the efficiency of the original TRS is unpredictable and not only depends on the ratio and concentration of the delivered transactivator and reporter plasmids, but also on the site of integration of the plasmids intra-chromosomally. Most reports using the TRS have at best managed to achieve 10- to 100-fold regulation with few studies exceeding this. For in vivo gene regulation, where absolute concentration of tetracycline is subject to individual's pharmacokinetics and pharmacodynamics, 100-fold regulation is less than desirable. In these instances, a 1000-fold regulation is far more practical, with the aim of reaching 100%, 10%, 1% and 0.1% gene regulation.

The original TRS also displays relatively high basal leakiness that is a function of the minimal CMV promoter of tRP and it is therefore cell-type dependent (Agha-Mohammadi and Hawkins, 1998). Several permutations have attempted to reduce this leakiness through the use of an alternative minimal promoter that simultaneously reduced maximal expression (Hoffmann, A., Villalba, M., Journot, L., and Spengler, D. (1997). A novel tetracycline-dependent expression vector with low basal expression and potent regulatory properties in various mammalian cell lines. Nucleic Acid Res. 25(5),1078-1079, incorporated herein by reference in its entirety) and a complicated set-up that required Dox-controlled transcriptional down-regulation by tetR-KRAB and up-regulation by rtTA within the same cell (Rossi, F. M., Guicherit, O. M., Spicher, A., Kringstein, A. M., Fatyol, K., Blakely, B. T., and Blau, H. M. (1998). Tetracycline-regulatable factors with distinct dimerization domains allow reversible growth inhibition by p16. Nat. Genet. 20,389-93; Forster, K., Helbl, V., Lederer, T., Urlinger, S., Wittenburg, N., and Hillen, W. (1999). Tetracycline-inducible expression systems with reduced basal activity in mammalian cells. Nucleic Acids Res. 27,708-710; both of which are incorporated herein in their entireties).

Finally, as a chimeric regulatable system, the TRS transactivators may have potential immunogenicity. Even though, gene expression and regulation have been followed for more than 6 months, with no detection of anti-tTA antibody (Bohl et al., 1997), both tetR and VP16 domains are foreign and potentially immunogenic.

To overcome these concerns, a “second generation tetracycline regulatable system” was constructed and characterized. As discussed in further detail below, the second generation tRPs were devised to reduce the basal expression of previously described pCMV*⁻¹.mGM-CSF, while enhancing the efficiency of regulation. By removing the upstream sequence of mGM-CSF, plasmid pCMV*⁻².mGM-CSF with reduced basal expression was constructed. Plasmid pCMV*⁻².mGM-CSF, which has the same minimal promoter as tRP, showed approximately 10-fold reduced basal expression compared to pCMV*⁻¹.mGM-CSF. Plasmid pCMV*⁻².mGM-CSF was selected as a control to represent the activity of the tRP.

To selectively enhance the maximal expression from this plasmid, the synergy between consecutive tTA molecules was maximized by reorganizing the tetO sites. The effects of distance and position of 2 tandem tetO sites on tTA synergy was investigated by varying the distance between the centers of 2 consecutive tetO sequences from 2 to 5½ helical turns in ½ turn increments. Maximal expression and thus synergy was achieved when the distance between the centers of the 2 tetO sites ranged from 21 to 52 bases. Within this range, maximal tTA synergy occurred when the central base(s) of the 2 consecutive tetO sites were separated by 2½, 3½ and 4 1/2 helical turns of the DNA and thus positioned on opposite surfaces of the DNA. This observation is consistent with a model that proposes a 0.4 turn unwinding of wild type tetracycline-resistant operon on binding of tetR molecules to the 2 tet operators. This results in positioning of the tetR molecules on opposite sides of the DNA despite a separation of 30 base pairs between the 2 tetO centers (Lederer, H., Tovar, K., Baer, G., May, R. P., Hillen, W., and Heumann, H. (1989). The quaternary structure of Tet repressors bound to the Tn10-encoded tet gene control region determined by neutron solution scattering. EMBO J. 8,1257-1263, incorporated herein by reference in its entirety). A similar unwinding of DNA by tTA can position all the VP16 transactivational domains on the same side of the DNA in 2tetO-26, -36, and -47 plasmids where maximal synergy may occur.

The results obtained from these experiments substantiate the transactivator synergy model of transcription. According to that model, transcriptional synergy occurs when closely aligned transactivators cooperate to initiate transcription. By optimizing tTA synergy through rotational alignment of the tTA molecules, the results presented herein support the model of transactivator synergy and further highlight the importance of positions of DNA binding sites in this process. The data obtained can be applied to optimize other existing regulatable systems, such as the progesterone-, ecdysone-, rapamycin-regulatable systems (Agha-Mohammadi and Lotze, 2000) by maximizing transactivator synergy through rotational alignment

To further enhance the maximal expression of the tRP, tTA synergy was augmented through the use of multiple tetO sequences. tTA synergy was progressively enhanced by increasing the number of tetO sites from 2 to 8, resulting in higher efficiencies of regulation. One plasmid, p8tetO-36.mGM-CSF, had a 5- to 10-fold higher regulation efficiency than the original tRP by attaining 5- to 10-fold higher maximal expression while conserving the basal expression. At a 1:1 ratio of transactivator to reporter plasmid, p8tetO-36.mGM-CSF consistently displays over a 1000-fold gene regulation. Compared to the original TRS, the second generation tRPs also displayed 5- to 10-fold improved efficiencies in 293 cells and NIH 3T3 cell lines (Data not shown). Similar findings have been noted on using the second generation tRPs in the context of the Tet-on system (See Example 6, below). Thus, the second generation tRPs overcome the relative inefficiency of the original tRP, especially for in vivo use. In the Examples, below, pCMV*⁻².mGM-CSF was used so that the efficiency of the new second generation tRP can be compared with the original tRP. The observed leakiness of the second generation tRP and the original tRP are similar in these experiments.

A number of shortened versions of the minimal tRP promoters were made having reduced basal leakiness while preserving the high efficiency of the second generation tRP. As with the tetracycline-regulatable system, other regulatable systems may benefit from inclusion of more than two DNA transactivator binding domains. An important application of the second generation tetracycline regulatable system is in construction of tetracycline-regulated replication-effective viruses. As an example, replication-effective adenoviruses that are controlled through regulation of the E1 gene of the adenovirus will be of importance in future cancer gene therapy.

In connection with the TRS, to overcome the inconsistent and unpredictable delivery of that system, a single positive feedback regulatory system was constructed based on the second generation tRP. Previously, it has been demonstrated (S. Agha-Mohammadi and Hawkins, R. E. (1998)) that a positive feedback regulatory system (pSiaIV) based on the original tRP, displays high levels of transgene expression on induction and low levels of basal expression on repression. A second generation PFRV II based on p8tetO-36.mGM-CSF, not only maintained the properties of the original PFRV, but also displayed higher regulation efficiencies of about 900-fold as a result of enhanced maximal expression. This appears to be the highest regulation that has ever been reported from a single tetracycline-controlled plasmid. The high degree of regulation and the ease of delivery of this vector will be of importance in in vivo studies and gene therapy, where predictable delivery and high regulation efficiency are particularly needed. Finally, to address the potential immunogenicity of tTA, we have developed 2 chimeric transactivators using the human p65 C-terminal residues in place of the herpes simplex virus VP16. Both tetR-p65²⁸¹ and tetR-p65³¹³ were functional and as effective as tTA in achieving high maximal expressions and regulation efficiencies of about 1000-fold.

In conclusion, the tetracycline regulatable system is one of the most valuable tools for controlling gene expression both in vitro and in vivo. This system, however, still suffers from a number of imperfections. These concerns are of exceptional importance in in vivo use of the system where ease of set-up and delivery, high regulation efficiency, and reduced immunogenicity are particularly needed. To address these issues, a second generation of tetracycline-regulated promoters and transactivators were devised. The second generation tRPs display enhanced regulation efficiency by increasing the maximal expression of the promoter by augmenting tTA synergy. These promoters sustain their regulation efficiency in the context of a single positive feedback regulatory vector that presents ease of delivery of the system for in vivo and gene therapy use. Finally, second generation transactivators utilizing human transactivational domains should reduce the potential immunogenicity of the original system.

Therefore, provided herein is a nucleic acid comprising in its most minimal sense two or more transactivator binding domains, such as tetO sequences, separated by a specific number of bases so that transactivators binding adjacent transactivator binding domains are rotationally aligned about the DNA helix. In the case of the tetO system, adjacent tetO sequences that are not bound by transactivator fall on opposite sides of the DNA helix and, when bound by transactivator are approximately aligned at their palindromic centers. A transactivator binding domain is a sequence to which a transactivator or transrepressor can bind and includes, without limitation, tetO sequences, GAL4-DNA binding sequences, ecdysone ECRE sequences and the rapamycin system's ZFHD1 sequences.

As used herein, a “turn” of the double helix is in reference to the relative rotational relationship between two bases in most DNA forms, with each 10-11.5 base separation between the two bases being a single “turn”, referring to a single 360° rotation of the DNA helix. Thus, a single turn is a separation of about 10 bases, 2 turns is about 21 bases and 2½ turns is about 26 bases, recognizing that the number of turns is a close approximation because a single turn corresponds roughly to a non-integer 10.4 bases.

In the tetracycline-regulatable system described herein, center nucleotides of adjacent tetO sequences are typically separated by 23 to 29, 33 to 39 or 43 to 50 bases, more typically by 25 to 27, 35 to 37 or 45 to 47 bases, and as provided in the Examples below, by 26, 36 or 46 bases.

By “tetO sequence” it is meant a tetracycline operator sequence. tetO sequences include A1, A2, B1, B2, C1, C2, D1, D2, E1 and E2 tetO sequences (native tetO sequences) and operable mutants thereof. tetO sequences, as described, for instance in U.S. Pat. No 6,138,954, FIG. 5, incorporated herein by reference, have palindromic features and typically have a single “central nucleotide” that is the center of the palindromic sequence. Mutants of the tetO sequence are known and are limited only by their ability to bind to a corresponding tetracycline repressor (tetR) protein either in native form or as a fusion protein with a eukaryotic transactivator peptide, such as herpes simplex VP16, Epstein-Barr virus (EBV) rta and human NF-κB p65 transactivators (tTA and rtTA). Mutant tetO sequences are described in Sizemore C., Wissmann A., Gulland U. and Hillen W. (1990). Quantitative analysis of Tn10 Tet repressor binding to a complete set of tet operator mutants. Nucl. Acid Res. 18(10):2875-2880.

A “fusion protein” refers to two or more polypeptides that are operatively linked and typically, but not necessarily, form a single polypeptide chain. Further, each operatively linked polypeptide in the fusion protein can serve its intended function and the fusion peptide can serve its intended function. Operative mutants of tetO sequences include native tetO sequences having insertions, deletions and substitutions that do not result in the inability of tetR, tTA or rtTA proteins to bind the tetO mutant either in the absence of tetracycline (a Tet-off system), as in the case of the tetR and tTA proteins, or in the presence of tetracycline (a tet-on system), as is the case with rtTA proteins.

Other transactivator DNA binding sequences are known and transactivators that bind those sequences also have been described. These include: the HSV VP16-GAL4 DNA-binding domain/mutant progesterone receptor (PR-LBD) transactivator of the progesterone-regulatable system; a heterodimeric complex of VpECR (a fusion of VP16, the N-terminal truncation of a mutant ecdysone receptor and the DNA-binding domain of the glucocorticoid receptor) and the retinoid X receptor in the ecdysone-regulatable system; and the ZFHD1 (DNA binding unit)-FKBP12 chimera in combination with the FRAP/NF-κB p65 chimera in the rapamycin-regulatable system (see, e.g., Agha-Mohammadi and Lotze, 2000).

As described herein, tetR is a protein that specifically binds a tetO sequence in the absence of tetracycline. Native tetR proteins are specific to each native tetO sequence. Thus a tetR that binds the A-type tetO sequence does not necessarily bind B-, C-, D- and E-type tetO sequences. A tetracycline transactivator (tTA) is a fusion protein between a tetR sequence and a eukaryotic transactivator. The tetR sequences in the fusion protein are substantially complete in that they include DNA binding- and tetracycline-binding domains as well as all peptide sequences necessary for tetracycline-regulatable binding to the tetO sequences. In the presence of tetracycline, tTA does not bind its corresponding tetO sequence. The tetR portion of the tTA fusion protein minimally includes all sequences necessary for binding of the tTA to the tetO sequence in the absence of tetracycline and the release (non-binding) of tTA from the tetO sequence in the presence of tetracycline.

The rtTA fusion protein contains the same transactivational domain as the tTA fusion protein, but contains a mutated tetR portion that, as opposed to the tTA protein, binds its corresponding tetO sequence in the presence of tetracycline and does not bind its corresponding tetO sequence in the absence of tetracycline. Details of tTAs (such as that encoded by pUHD 15-1) and rtTAs (such as that encoded by pUHD 17-1) are provided in Example 1 of U.S. Pat. No.6,136,954 (Columns 45 and 46, incorporated herein by reference). Mutant tTA and rtTA proteins are described in the literature, for instance in Example 7 of U.S. Pat. No. 6,138,954 (Columns 54-62, incorporated herein by reference) and in U.S. Pat. No. 6,271,341 (incorporated herein by reference in its entirety for its disclosure of tTA fusion proteins having graded transactivational potential).

In contrast to the above-described transactivators, transrepressors may be useful in the regulatable systems described herein. For instance, Deuschle, U., Meyer, W. K. H., and Thiesen, H. J. (1995) describe a fusion protein consisting of a tetR unit and the KRAB repressor domain of the human Kox1 zinc finger protein. Therefore, in addition to the transactivator fusion proteins, transrepressor proteins may be used in the optimized regulatable systems described herein.

The nucleic acid described herein minimally includes 2 and typically includes 2-8 tetO sequences distanced from each other as described above so no adjacent tetO central nucleotides are in rotational alignment when the nucleic acid is in B form. Typically, each adjacent pair of tetO sequences are spaced from each other the same distance as other adjacent pairs of tetO sequences in the same nucleic acid sequence. The nucleic acid may be only long enough to include the plurality of tetO sequence, optionally including a linker or a recombination sequence at its ends, and, therefore, is useful as an insert for a gene expression system. The insert would be placed an appropriate distance from and location with respect to a promoter so that it is operably linked to that promoter. The addition of linkers or recombination sequences to the ends of the insert would facilitate attachment of the insert to other regulatory and coding sequences.

A linker is a short nucleic acid sequence that includes a restriction site that typically is not present in the insert and are typically are restriction sites for rare-cutting restriction endonucleases that are single- or dual-cutters for the sequence to which the insert is to be attached. A polylinker is a linker having more than one restriction site. A recombination sequence is a sequence that when used with particular cloning systems, can direct by a recombination event insertion of a nucleic acid flanked by two recombination sequences into another nucleic acid containing a co-reacting recombination sequence or sequences, such as a CRE-LoxP system (for example, the CREATOR™ cloning system commercially available from Clontech and GATEWAY system from Invitrogen).

As used herein, the term “operably linked” means that elements that are connected in a manner such that each element can serve its intended function and the elements, together can serve their intended function. In reference to elements that regulate gene expression, “operatively linked” means that a first regulatory element or coding sequence in a nucleotide sequence is attached and oriented in relation to a second regulatory element or coding sequence in the same nucleic acid so that the first regulatory element or coding sequence operates in its intended manner in relation with the second regulatory element or coding sequence. In relation to the present invention, a tetO sequence is operatively linked to a promoter to form a tRP that when incorporated into a complete gene, including operably linked tetO sequences, a promoter, a coding sequence and a polyadenylation (polyA) signal, the tetO sequences can be used to control expression of the coding sequence in the presence of tTA and rtTA proteins. A promoter is operably linked to a coding sequence to promote transcription of that coding sequence. A regulatory element is a nucleotide sequence that controls expression of, a coding sequence, alone, or in combination with other nucleotide sequences or trans factors, such as repressor proteins like tetR, activator proteins, such as tTA and rtTA, polymerases and polymerase complex proteins such as initiator proteins. Regulatory elements include, without limitation, operators, enhancers, promoters and polyA signals. By “sufficient regulatory elements to control gene expression” it is meant that a nucleic acid includes any operatively linked regulatory elements needed for the intended expression of the gene, typically including minimally a promoter and a polyA signal, and, in the context of the present disclosure, tetO sequences.

A coding sequence is a sequence that either encodes a protein, therefore including an open reading frame or a functional RNA, such as, without limitation, an anti-sense sense RNA or a ribozyme. The coding sequence may include one or more intron sequences.

The nucleic acid described herein typically contains a promoter operably linked to the transactivator binding domains to form a regulatable promoter (RP), or tRP when the domains are tetO sequences. Broadly defined, a “promoter” is a DNA sequence that determines the site of transcription initiation for an RNA polymerase. Because the expression system described herein includes sequences for binding a transactivator or transrepressor protein, such as the tTA or rtTA proteins, that controls certain promoters, the only limitations places on the provided definition of “promoter” is that the “promoter” is selected to be responsive to transcriptional regulation by a the transactivator or transrepressor when that protein is bound to the transactivator binding domains operably linked to the promoter. This requirement, in many cases, is not very stringent in that many known transactivators can enhance transcription from many different promoters. For instance, herpes simplex virus VP-16, EBV rta and human NF-κB p65 transactivators enhance transcription from many known eukaryotic promoters, including viral promoters, notably the CMV promoter as is described in the literature with regard to tetracycline-responsive systems, and as is described herein.

A promoter can be, without limitation, constitutive, tissue-specific, or otherwise controllable. Examples of promoters include, without limitation, the CMV, HSV-Tk, human β-actin, muscle creatinine kinase (MCK), amylase and albumin promoters. In any case, any promoter can be tested readily for its effectiveness in the tetracycline-responsive expression system described herein by substitution for the minimal CMV promoter described herein. Modified promoters also may be used, including insertion and deletion mutation of native promoters and combinations or permutations thereof. One example of a modified promoter is the “minimal CMV promoter” the sequence of which is provided in Example 5 (SEQ ID NO: 21), below.

An “expression cassette” including the above-described RP, including a promoter, such as a viral promoter, for instance a CMV or CMV minimal promoter, and a polyA sequence. A linker, preferably a polylinker containing multiple single-cut restriction sites, or a recombination sequence, such as LoxP, is placed between the promoter and polylinker facilitating insertion of, and thereby operable linkage of any coding sequence to the promoter and polyA site. The expression cassette typically is propagated as an autonomously replicating genetic unit, most typically as a plasmid, more typically as a ColE1 plasmid containing an E. coli replication origin and one or more selectable markers, for instance antibiotic resistance genes, for selection in bacteria and/or in eukaryotic cells.

The above-described RP, typically a tRP, may be in the form of a gene in which the RP is operably linked to a coding sequence and a polyA signal, may be operably linked to 1) sequence(s) that permit packaging of the gene into a virus particle, 2) sequence(s) that permit autonomous eukaryotic propagation of the plasmid, such as viral origins of replication and/or 3) sequence(s) that permit or facilitate integration of the gene containing the tetO/promoter sequence into a eukaryotic genome. Much research activity is devoted to the use of recombinant viral vectors such as retrovirus, adenovirus, retrovirus/adenovirus and Adeno-associated virus vectors to facilitate the transformation of cells with a desired gene, such as for therapeutic applications. The literature is replete with examples of these systems and their use in transferring genes to cells. The literature also is replete with examples of the successful transfer of genes containing first generation tetracycline-regulatable elements to eukaryotic cells, both in vitro and in vivo, by a variety of viral-based vectors. Description of some viral systems is provided in U.S. Pat. No. 6,271,341, column 10, lines 36 to 63,-which is incorporated herein by reference. Examples of these viral vectors include: 1) the tetracycline-regulatable gene or expression cassette flanked by two retroviral LTR sequences (see, for example, S. Agha-Mohammadi and Lotze, M. T. (1998). “Regulatable Systems: Applications in Gene Therapy and Replicating Viruses” J. Clin. Investigation 105(9):1177-1183, incorporated herein by reference in its entirety; U.S. Pat. No. 6,133,027, specifically columns 17 to 32 thereof, which is incorporated herein by reference; and U.S. Pat. No. 5,965,440, entitled “Controlled Gene Product Delivery From A Regulatable Retroviral Vector”, which is incorporated by reference in its entirety) 2) the tetracycline-regulatable gene or expression cassette flanked by two Adeno-associated virus ITR sequences (see, U.S. Pat. No. 5,753,500, incorporated herein by reference in its entirety for its description of recombinant AAV technology); and 3) the tetracycline-regulatable gene or expression cassette flanked by adenovirus (see, for example, S. Agha-Mohammadi and Lotze, M. T. (1998)). Retroviral and Adenoviral tetracycline-regulatable expression systems are commercially available from Clontech.

In the tetracycline system, the nucleic acid also may contain a gene for the production of the tTA or rtTA protein, most preferably including a sequence encoding the tTA or rtTA protein operably linked to a promoter which, in turn, is operably linked to the tetO sequences, but oriented in a direction opposite that of the first tetracycline-regulatable expression cassette or gene. This is, in one embodiment, a second generation version of the single self-contained vector tetracycline-regulatable expression systems described in the literature, such as in S. Agha-Mohammadi and Lotze, M. T. (1998) and as further described in Example 6, below. This second generation version substitutes the novel second generation tRP described herein for the first generation tRP. The tetO sequences bind tTA or rtTA fusion proteins, which control expression bi-directionally from both the tTA or rtTA gene and the first gene described above containing any coding sequence. Similar constructs may be made to produce transactivators or transrepressors for regulatable systems other than the TRS.

In one variation of this single vector embodiment, the coding sequence for tTA or rtTA is substituted with a different coding sequence, permitting bi-directional expression of two different genes, such as a marker and another gene that cannot be easily detected. Such vectors, using the first generation tRP are commercially available from Clontech, and are designated pBI TET vectors. Transactivators, such as tTA and rtTA, must be provided in these systems either by co-transfection of a plasmid containing the tTA or rtTA genes, or by other means.

The transactivator or transrepressor proteins also may be provided by a gene that has been established in a cell line. Cell lines transformed with the tTA or rtTA genes are available commercially from Clontech. These cell lines are especially useful when a biocatalyst or producer cell line is desired. Cells transformed by the transactivators can be transformed with a gene under control of the second generation tRP described herein to produce a desired biocatalyst or to produce a desired gene product, for instance in biopharmaceutical production.

As described in the literature in connection with the first generation regulatable gene expression systems, the second-generation tetracycline-regulatable gene expression systems described herein may be included in a variety of vector systems and may be used in any application that the first generation systems may be used. The expression cassette, gene or single-vector embodiments may be placed in any vector, such as, without limitation, plasmids, phagemids, cosmids, phage and viral vectors, insect vectors, yeast vectors (such as a yeast artificial chromosome), plant vectors (such as Ti plasmids) and combinations thereof as are known in the art. Methods for producing transgenic or homologous or enzyme-assisted recombination non-human animals (such as mice or farm animals, including goats, sheep, pigs, or cows) and plants are known and conventional in the art and are described, for instance in U.S. Pat. No. 5,589,362, columns 15 and 16, incorporated herein by reference.

As used herein, a “cell” may be any cell in which the nucleic acid can either be propagated or can be used to introduce a foreign gene into the cell to either temporarily or permanently transform the cell. The nucleic acid is typically propagated in bacterial cells, most commonly as a ColEI plasmid in an E. coli cell. However, other systems for propagation of vectors are known, including without limitation other bacteria, yeast, insect cells and mammalian cells. Mammalian cells may be preferred if the vector is a viral vector such as an adenovirus, a retrovirus or an adeno-associated virus. The cells to be transformed, or transformed cells, typically are eukaryotic cells and can vary according to the desired application. These cells include: 1) cultured cells, such as HeLa cells optionally transformed with transactivator or transrepressor gene, such as a tTA or rtTA gene, typically for use in research, bioconversion and biocatalysis applications or ex vivo uses, such as in therapeutic applications; 2) cells in vivo, typically for therapeutic applications and in vivo cells transformed with the nucleic acid either by delivery to the organism or by somatic transformations, including transgenic or recombination methods, in which case, the cells range from non-mammalian cells, such as, plant, insect or non-mammalian vertebrate cells to mammalian cells.

Following from the above, provided is a method for preparing an expressible gene that includes the step of ligating and thereby operably linking a nucleic acid including a coding sequence to a nucleic acid including a second generation tRP as described herein.

Also provided is a method for modulating expression from a regulatable promoter including two or more transactivator binding domains. The method includes the step of modifying the distance between the two or more domains to achieve a desired degree of regulation efficiency, that is to vary the maximal and basal transcription rates.

Also provided is a method for expressing a gene in a cell. The method includes the steps of: introducing a nucleic acid into the cell, the nucleic acid comprising a second generation RP, such as the tRP described herein operably linked to a coding sequence, and expressing a transactivator protein, such as a tTA or rtTA protein, in the cell, such that expression of the coding sequence is activated either when the cell is contacted with an inducer, such as tetracycline, or an analog thereof, or by the removal of the inducer from contact with the cell. The nucleic acid may be introduced into the cell by any method, including, without limitation, viral transduction, liposome transfection, calcium phosphate precipitation, DEAE-dextran transformation, particle bombardment, microinjection, and endocytosis.

Although the TRS systems described herein are described as known as tetracycline-regulatable, tetracycline analogs may be as useful, or more useful than tetracycline for the purpose of binding tetr, or transactivator or transrepressor derivatives thereof. As used herein and as is known in the art, doxycycline may be preferred to tetracycline in its use in binding to tetR or derivatives thereof. Other useful pharmaceutically acceptable tetracycline analogs include: chlortetracycline, oxytetracycline, demethylchloro-tetracycline, methacycline, doxycycline and minocycline. Thus, a method is provided for controlling expression of a tetracycline-regulatable gene including the step of contacting a cell containing a nucleic including the tetracycline-regulatable gene with one of chlortetracycline, oxytetracycline, demethylchloro-tetracycline, methacycline, doxycycline and minocycline.

One practical application of the expressible systems described herein is, without limitation, the ex vivo transformation of cells for therapeutic or other purposes. For instance, dendritic cells [DCs] may be isolated by known methods and transformed with a gene as described above. DCs are capable of migrating into tissues, taking up antigen and then migrating into lymph nodes. This property confers on them an ability to deliver cytokines, genes, drugs or prodrugs to tissues. As such, DCs may be transformed ex vivo with a regulatable gene as described herein and re-introduced into a patient in order to target delivery of the regulatable gene to a desired location. DCs target sites of inflammation, which may be a condition of the patient, or which may be induced by injury or other agents. Chronic inflammation including cancer, chronic viral disease, autoimmunity, or allograft rejection may be accessible to such approaches. Homing of DCs also may be enhanced by expression on the surface of the dendritic cell of a homing receptor or “addressin” specific for tissue or organs. An addressin may be generated by phage-display screening processes as are known in the art and the sequences encoding the addressin might be expressed from a second transformed gene, including from a second coding sequence in the above-described bi-directional tRP system.

The gene to be inserted into the dendritic cells may be one of any class of gene that would be useful, including: cytokines for targeted secretion; antigen for targeted presentation as, for instance, a vaccine; a co-stimulatory surface-expressed molecule; an enzyme that can convert an inactive prodrug or other inactive compound to an active drug or compound for targeted drug delivery; and a system including a dendritic cell that is pre-loaded with a drug in vesicles (for instance, by liposome or exosome delivery) and which contains an inducible gene that causes release of the drug after the dendritic cell homes to its target site. In certain cases, a non-leaky regulatable promoter, such as the promoter of Example 5, below, may be preferred.

### EXAMPLES

A. Methods

Plasmids and DNA Manipulation

The plasmid pCMV*⁻¹.mGM-CSF has been described before (Agha-Mohammadi et al., 1997). This plasmid contains about 50 bases upstream of the mGM-CSF cDNA and downstream to the minimal CMV promoter of tRP. The plasmid pUHD 15-1 (Clontech) contains the tTA transactivator gene transcribed from the CMV IE promoter (Gossen and Bujard, 1992). To construct a control plasmid that represents the minimal CMV promoter of tRP, the sequence between the transcriptional initiation codon of mGM-CSF and the minimal CMV promoter was deleted in pCMV*⁻².mGM-CSF. To reduce the basal leakiness of the minimal CMV promoter, we have constructed several vectors with shortened version of the minimal CMV promoter. Plasmid p8tetO-16.mGM del R1-SacII has a deletion of sequence corresponding to bases 439 to 449 of pUHD 10-3 (Gossen and Bujard, 1992). This deletion removes 10 bases of the distal end of the minimal CMV promoter. The plasmid p8tetO-16.mGM del R1-Bbs1 has a deletion of sequence corresponding to bases 414 to 449 of pUHD 10-3. This deletion removes 35 bases of the distal end of the minimal CMV promoter. Plasmid p8tetO-16.mGM del KpnI-StuI has a deletion of sequence corresponding to bases 304 to 336 of pUHD 10-3. This deletion removes 32 bases upstream of the TATA box. The replacement of the whole minimal CMV IE promoter with a sequence extending from 6 bases upstream of the TATA box to 36 bases downstream of TATA (bracketed in the sequence provided in Example 5, below), resulted in construction of a micro CMV IE promoter in p8tetO-16.TATAII.mGM.

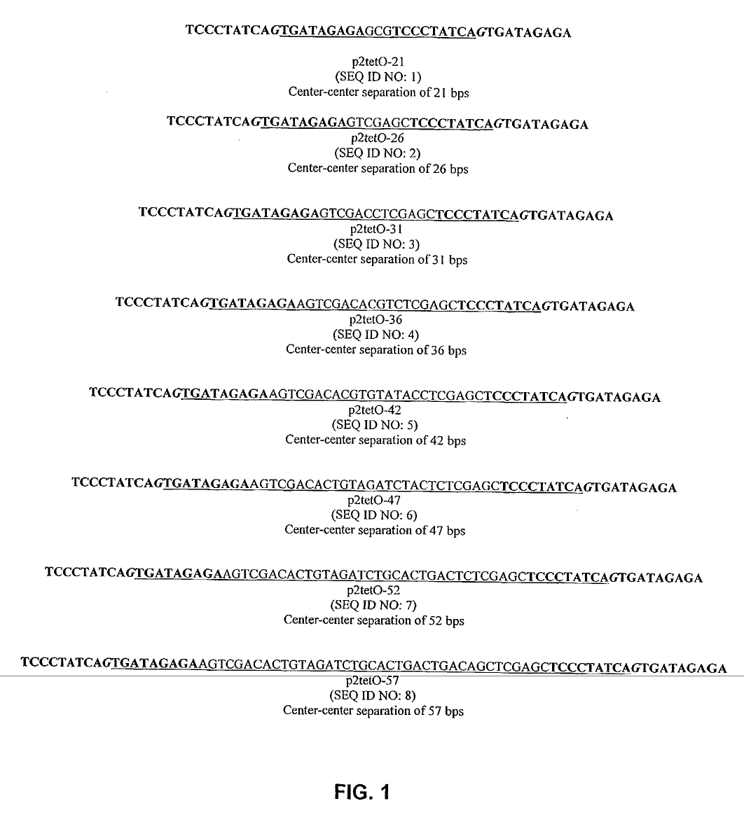

To construct p1tetO.mGM-CSF, the oligonucleotides 5′ TCGAGCTCCCTATCAGTGATAGAGAAGGTAC 3′ (SEQ ID NO: 9) and 5′CTTCTCTATCACTGATAGGGAGC 3′ (SEQ ID NO: 10) were annealed and ligated into XhoI/KpnI digested pCMV*⁻².mGM-CSF. The annealed oligonucleotide corresponds to the sequence of a single tetO₂ with XhoI and KpnI overhangs. Plasmids, p2tetO-21.mGM-CSF, p2tetO-26.mGM-CSF, p2tetO-31.mGM-CSF, p2tetO-36.mGM-CSF, p2tetO-42.mGM-CSF, p2tetO-47.mGM-CSF, p2tetO-52.mGM-CSF, and p2tetO-57.mGM-CSF were constructed by placing a second tetO₂ oligonucleotide, between XhoI and AatII sites, at 3, 8, 13, 18, 24, 29, 34, and 39 bases away from the first tetO₂ element, respectively. For each plasmid, the number that follows the tetO denotes the base pair separation between the central bases of the 2 consecutive tetOs. Sequences of the XhoI/AatII inserts for each plasmid are provided in FIG. 1. Plasmids p4tetO-26.mGM-CSF, p4tetO-36.mGM-CSF, and p4tetO-47.mGM-CSF were constructed by digesting the respective 2tetO plasmids with AatII and BamHI to remove the 2tetO sites/minimal promoter/mGM-CSF cassette.

Plasmid p4tetO-26.mGM-CSF was constructed by placing blunt-ended 2tetO-26/minimal promoter/mGM-CSF insert into blunt-ended KpnI/BamHI cut p2tetO-26.mGM-CSF backbone and confirmed by restriction digest and sequencing for the plasmid with 4 tetO sites each separated by 8 bases by sequencing.

To construct p4tetO-36.mGM-CSF, the 2tetO-36/minimal promoter/mGM-CSF insert was ligated into KpnI/BamHI-cut p2tetO-36.mGM-CSF backbone via random linker-18 (5′CCCGGAGACGT 3′ (SEQ ID NO: 11) and 5′CTCCGGGGTAC 3′ (SEQ ID NO: 12); Life Technologies) with restriction overhangs for KpnI and AatII. Similarly, the 2tetO-47/minimal promoter/mGM-CSF insert was ligated into KpnI/BamHI cut p2tetO-47.mGM-CSF backbone via a random linker (5′CCCGGAATCGAGTCATCGACGT 3′ (SEQ ID NO: 13) and 5′CGATGACTCGATTCCGGGGTAC 3′ (SEQ ID NO: 14)) with restriction overhangs for KpnI and AatII.

Plasmid p6tetO-36.mGM-CSF was constructed by inserting the 2tetO-36/minimal promoter/mGM-CSF cassette into the KpnI/BamHI digested p4tetO-36.mGM-CSF using linker-18. Similarly, p8tetO-36.mGM-CSF was constructed by inserting the 2tetO-36/minimal promoter/mGM-CSF cassette into the KpnI/BamHI digested p6tetO-36.mGM-CSF using linker-18. Plasmid PFRVII was constructed by removing tTA from pCI.tTA.neo (Agha-Mohammadi and Hawkins, 1998) with AatII and DraIII. The blunt-ended fragment was then ligated into the blunt-ended, alkaline phosphatase-treated AatII site of p⁸tetO-36.mGM-CSF. Plasmids with tTA and mGM-CSF oriented in opposite directions were selected.

To construct pCMVtetR-Rta, pUHD 15-1 was cut with EcoRI and AvaII and a 624 base pair fragment representing tetR was isolated. This fragment, together with a synthetic linker, were ligated into EcoRI/BamHI cut pUHD 15-1 to form pCMVtetR-Bsu36I. The synthetic linker, comprising of 5′GTCCCTAAGGTCG 3′ (SEQ ID NO: 15) and 5′GGATTCCAGCCTAG 3′ (SEQ ID NO: 16), placed a unique Bsu36I restriction site distal to tetR. The plasmid pMH212 (a gift of Mary Hardwick, Hardwick, J. M., Tse, L., Applegren, N., Nicholas, J., and Veliuona, M. J. (1992). The Epstein-Barr virus R transactivator (Rta) contains a complex, potent activation domain with properties different from those of VP16. J. Virol. 66,5500-5508) was digested with Bsu36I and Bg1II to remove the C-terminal 520-605 amino acids of Rta. This fragment was inserted in frame with the coding sequence of tetR into Bsu36I/BamHI cut pCMVtetR-Bsu36I. Plasmid pCMVtetR-p65²⁸¹ was constructed by removing amino acids 281-551 of NF-κB p65 (Ballard, D. W. et al. (1992). The 65-kDa Subunit of human NF-κB functions as a potent transcriptional activator and a target for v-Rel-mediated repression. Proc. Natl. Acad. Sci. USA 89:1875-1879; Schmitz, M. L. et al. (1994). Structural and Functional Analysis of the NF-κB p65 C terminus. J. Biol. Chem. 269(41):25613-25620) of pCMV4-p65 (kindly provided by W Greene) with EcoRI and BamHI. This was then ligated into Bsu36I/BamHI cut pCMVtetR-Bsu36I via a serine/glycine/glycine linker using the annealed oligonucleotides 5′TAAGTGGCGGGG 3′ (SEQ ID NO: 17) and 5′GATTCCCCGCCAC 3′ (SEQ ID NO: 18) with overhangs for Bsu361 and EcoRI.

Plasmid pCMVtetR-p65³¹³ was constructed by removing amino acids 313-551of NF-κB p65 with BspHI and BamHI. This was then ligated into Bsu361/BamHI cut pCMVtetR-Bsu36I via a serine/glycine/glycine linker using the annealed oligonucleotides 5′TAAGTGGCGGGAT 3′ (SEQ ID NO: 19) and 5′CATGATCCCGCCAC 3′ (SEQ ID NO: 20) with overhangs for Bsu361 and BspHI. The sequences of all constructed plasmids were verified by automated sequencing.

Cell Culture, Transfections, and Reporter Gene Assay

HeLa cells were maintained in 10% fetal bovine serum in Dulbecco's modified Eagle's medium. Transfection of purified plasmid DNA into the cell lines was carried out using a modified calcium phosphate precipitation protocol. To ensure similar transfection efficiencies among wells transfected with the same plasmid, a single precipitate was prepared and divided among the wells. To monitor the efficiency of the transfections, β-galactosidase expression from pCH1 10 (Pharmacia) was assessed as described previously (Agha-Mohammadi and Hawkins, 1998). Generally the difference between the number of cells stained was within 1.0- to 1.3-fold between the wells for a cell-type. However, when the difference was more than 1.3-fold, the experiment was repeated. Transfected cells were maintained in culture medium with or without 1 μg/ml of doxycycline (Dox) at the time of transfection and thereafter. On daily basis, the culture medium was replaced with fresh medium with or without Dox. At 60 hours, a sample of the supernatant was removed for quantification of mGM-CSF by ELISA (R+D) as previously described (Agha-Mohammadi and Hawkins, 1998). Each experiment was at least repeated 3 times.

### Example 1

Optimizing Maximal Expression of Tetracycline-Regulated Promoter

To reduce the basal expression of pCMV*⁻¹.mGM-CSF, the intervening sequence between mGM-CSF cDNA and the minimal CMV promoter of tRP was removed to form pCMV*⁻².mGM-CSF and pCMV*⁻³.mGM-CSF. Maximal and basal expressions from this plasmid was then compared with those of pCMV*⁻¹.mGM-CSF in co-transfection studies. As seen in Table 1, below, both the maximal and basal expressions of pCMV*⁻².mGM-CSF were reduced over 5-fold compared to those of pCMV*⁻¹.mGM-CSF Plasmid pCMV*⁻².mGM-CSF, which contains the native tRP was then used to optimize tTA transactivational synergy by repositioning tetO arrangement. The effect of distance and position of tandem tetO sequences on tTA transactivational synergy was evaluated by varying the minimum distance between the central bases of 2 consecutive tetO sequences from 2 to 5½ helical turns at ½ turn increments. The plasmids, p2tetO-21.mGM-CSF, p2tetO-26.mGM-CSF, p2tetO-31.mGM-CSF, p2tetO-36.mGM-CSF, p2tetO-42.mGM-CSF, p2tetO-47.mGM-CSF, p2tetO-52.mGM-CSF, and p2tetO-57.mGM-CSF were then compared with original pCMV*⁻¹.mGM-CSF and parental pCMV*⁻².mGM-CSF in co-transfection studies. For each plasmid, the number that follows the tetO denotes the base pair separation between the central bases of the 2 consecutive tetOs. The results of this experiment are provided in Table 1, below.

TABLE 1

| | | | Fold |
|---|---|---|---|
| Plasmid | Dox− | Dox+ | Regulation |
| pCMV*-1mGM | 135230 | 375 | 360 |
| pCMV*-2mGM | 38595 | 63 | 612 |
| p2tetO-21.mGM | 35674 | 84 | 423 |
| p2tetO-26.mGM | 71350 | 60 | 1195 |
| p2tetO-31.mGM | 33405 | 66 | 508 |
| p2tetO-36.mGM | 53120 | 49 | 1086 |
| p2tet0-42.mGM | 20690 | 81 | 254 |
| p2tet0-47.mGM | 72482 | 80 | 908 |
| p2tet0-52.mGM | 32910 | 77 | 427 |
| p2tet0-56.mGM | 11942 | 58 | 206 |

HeLa cells in 6-well plates were co-transfected with 1.5 μg of pCMV*⁻¹.mGM-CSF, pCMV*⁻².mGM-CSF, p2tetO-21.mGM-CSF, p2tetO-26.mGM-CSF, p2tetO-31.mGM-CSF, p2tetO-36.mGM-CSF, p2tetO-42.mGM-CSF, p2tetO-47.mGM-CSF, p2tetO-52.mGM-CSF, or p2tetO-57.mGM-CS, and 1.5 μg of pUHD 15-1, together with 0.5 μg of pCH110 as a control for assessing transfection efficiency. Experiments were repeated 4 times to establish a pattern.

As can be seen from Table 1, the maximal expression and thus efficiency of the 2tetO promoters is dependent on the separation distance and position of the 2 consecutive tetO sites. Generally, the constructed plasmids, however, displayed comparable basal expressions. Plasmid p2tetO-21.mGM-CSF has 2 tetO sites separated by 3 base pairs. The central base of each palindromic tetO site is therefore positioned exactly on the same side of the DNA, considering a 10.4 base for each helical turn of B-DNA and a 9 base flank on each side of the central base of tetO. This plasmid displayed the lowest maximal expression which probably reflects hindrance to the attachment of tTA molecules to the adjacent tetO sites. Maximal expression is best achieved when the distance between centers of the 2 tetO sites ranged from 26 to 52 bases. Within this range, maximal expression varies more significantly with the positional relations of the 2 tetO sites, rather than simply the distance between the sites. Plasmids 2tetO-26, 2tetO-36, and 2tetO-47 display the highest maximal expression and the most efficient regulation in response to Dox. In these plasmids, the central bases of the 2 tetO sites are separated by 2½, 3½, and 4½ helical turns and are therefore positioned on opposite sides of the DNA. Also, the efficiency of plasmids 2tetO-31, 2tetO-42, and 2tetO-52 are similar and as a group, uniformly lower than those of 2tetO-26, 2tetO36, and 2tetO-47 plasmids. In these plasmids, the central bases of the 2 tetO sites are separated by 3, 4, or 5 turns of the DNA helix, respectively. The insignificant differences in the efficiencies of 2tetO-26, 2tetO-36, and 2tetO-47 plasmids highlights the overwhelming importance of positional relation of the tetO sites; rather than the distance of separation.

Plasmids p2tetO-26.mGM-CSF, p2tetO-36.mGM-CSF, regulation efficiencies which is about 2-fold higher than that achieved with pCMV*⁻².mGM-CSF). This appears to be the highest regulation ever obtained when equal ratios of transactivator and reporter plasmids are co-transfected. It is interesting to note that the maximal expression achieved from p2tetO-26.mGM-CSF, p2tetO-36.mGM-CSF, or p2tetO-47.mGM-CSF is consistently higher than that of pCMV*⁻².mGM-CSF in repeated studies. This indicates that positional interaction of tetO sites is far more important than the number of tetO sites per se. Plasmid pCMV*⁻².mGM-CSF has 7 tetO sequences with tetO center-center separations of 41 bases pairs, and thus resemble p2tetO-42.mGM-CSF in having tetO central bases that are positioned on the same side of the DNA.

From the results obtained, the locations of the 2 tetO sites on opposite sides of the DNA appear to play an important role in enhancing synergy of VP16 transactivator of tTA molecules. To further validate this finding and to determine the role of tTA on this observation, the effect of another chimeric transactivator, tetR-Rta, on the constructed plasmids were investigated. Rta is a transcriptional activator of EBV with a C-terminal transactivation domain (Hardwick et al., 1988). Even though, the maximal expressions obtained from tetR-rta were markedly lower relative to tTA, a very similar pattern of regulation efficiency was observed, as can be seen in Table 2, below. This confirms that the observed pattern of regulation efficiency is not related to the nature of the transactivator and is essentially a property of the reconstructed promoters.

TABLE 2

| | | | Fold |
|---|---|---|---|
| Plasmid | Dox− | Dox+ | Regulation |
| p2tetO-21.mGM | 2600 | 36.6 | 71 |
| p2tetO-26.mGM | 5820 | 29.5 | 197 |
| p2tetO-31.mGM | 3360 | 25.45 | 132 |
| p2tetO-36.mGM | 5950 | 21.8 | 272 |
| p2tetO-42.mGM | 2800 | 30.4 | 92 |
| p2tetO-47.mGM | 4790 | 27.3 | 175 |
| p2tetO-52.mGM | 2074 | 28.2 | 73 |

HeLa cells in 6-well plates were co-transfected with 1.5 μg of p2tetO-21.mGM-CSF, p2tetO-26.mGM-CSF, p2tetO-31.mGM-CSF, p2tetO-36.mGM-CSF, p2tetO-42.mGM-CSF, p2tetO-47.mGM-CSF, or p2tetO-52.mGM-CSF and 1.5 μg of pCMVtetR-Rta, together with 0.5 μg of pCH110 as a control for assessing transfection efficiency. Experiments were repeated 2 times to establish a pattern. For both experiments, the final mGM-CSF value represents the average of 6 samples. The standard error of each average was consistently less than 10% of the final value.

### Example 2

Second Generation Tetracycline-Regulated Promoters

The results obtained with the 2tetO plasmids indicated that the maximal expression of tRPs can be enhanced by modifying the arrangement of the tetO sequences. To further enhance the regulation efficiency of the new tRPs, we attempted at increasing the maximal expression of 2tetO-26, 2tetO-36, and 2tetO-47 vectors by using multiple tetO sites. Since the efficiencies of 2tetO-26, 2tetO-36, and 2tetO-47 were not significantly different in several repeated studies, plasmids with 4 tetO elements were constructed based on these vectors. In these promoters, center-center separation of consecutive tetOs were set at 26, 36, or 46 random base pairs, respectively. The plasmids were compared relative to pCMV*⁻².mGM-CSF in co-transfection studies.

As seen in Table 3, below, all of these promoters function comparably and efficiently, again confirming the importance of positional interaction. The 4tetO promoters achieve a higher maximal expression while maintaining the high regulation efficiency achieved with the 2tetO promoters.

TABLE 3

| | | | Fold |
|---|---|---|---|
| Plasmid | Dox− | Dox+ | Regulation |
| pCMV*-2mGM | 30380 | 66 | 444 |
| p4tetO-26.mGM | 156300 | 68 | 2319 |
| p4tetO-36.mGM | 116200 | 38 | 3058 |
| p4tetO-47.mGM | 83960 | 42 | 2008 |

HeLa cells in 6-well plates were co-transfected with 1.5 μg of pCMV*⁻¹.mGM-CSF, p4tetO-26.mGM-CSF, p4tetO-36.mGM-CSF, or p4tetO-47.mGM-CSF and 1.5 μg of pUHD 15-1, together with 0.5 μg of pCH110 as a control for assessing transfection efficiency.

Based on its slight superior efficiency (not statistically significant though), 4tetO-36 promoter was selected for further development. To further increase the maximal expression and the regulation efficiency of these new tRPs, plasmids with 6 and 8 tetO sites separated by 36 base pairs were next developed and compared to pCMV*⁻².mGM-CSF in co-transfection studies. The results obtained indicate that the maximal expression of these tRPs enhance as the number of tetO sites increases. Maximal expression, however, appears to reach a plateau with 4 tetO sites and further tetO sites do not enhance expression significantly. The maximal expressions from p4tetO-36.mGM-CSF, p6tetO-36.mGM-CSF, and p8tetO-36.mGM-CSF are significantly higher than the maximal expression from pCMV*⁻².mGM-CSF despite displaying comparable basal expressions, as shown in Table 4. These promoters are termed “second generation tetracycline-regulated promoters.”

TABLE 4

| | | | Fold |
|---|---|---|---|
| Plasmid | Dox− | Dox+ | Regulation |
| pCMV*⁻²mGM | 64460 | 140 | 306 |
| p2tetO-36.mGM | 259200 | 377 | 688 |
| p4tetO-36.mGM | 434900 | 368 | 1180 |
| p6tetO-36.mGM | 414700 | 363 | 1142 |
| p8tetO-36.mGM | 427900 | 250 | 1704 |

HeLa cells in 6-well plates were co-transfected with 1.5 μg of pCMV*⁻².mGM-CSF, p2tetO-36.mGM-CSF, p4tetO-36.mGM-CSF, p6tetO-36.mGM-CSF, or p8tetO-36.mGM-CSF, and 1.5 μg of pUHD 15-1, together with 0.5 μg of pCH110 as a control for assessing transfection efficiency. For both experiments, the final mGM-CSF value represents the average of 6 samples. The standard error of each average was consistently less than 10% of the final value. Experiments were repeated several times to establish a pattern.

### Example 3

Second Generation Tetracycline-Regulated Promoter Functions Efficiently as a Single Positive Feedback Regulatory Vector

To investigate the functionality of the second generation tetracycline-regulated promoter in the context of a single plasmid, a positive feedback regulatable system (PFRS) (Agha-Mohammadi and Hawkins, 1998) was constructed based on p8tetO=36.mGM-CSF. The plasmid, PFRV II, was then compared with co-transfected pCMV*⁻¹.mGM-CSF and pUHD 15-1, and p8tetO-36.mGM-CSF and pUHD 15-1. PFRS is made up of a modified bi-directional promoter driving the expressions of tTA and mGM-CSF reporter gene. In eukaryotic cells, both tTA and mGM-CSF will be produced at basal levels. Subsequently, tTA engages in a positive feedback loop up-regulating its promoter bidirectionally. The system offers increased sensitivity to tetracycline repression by a dual mechanism, i.e. terminating tTA transcription and causing its inactivation.

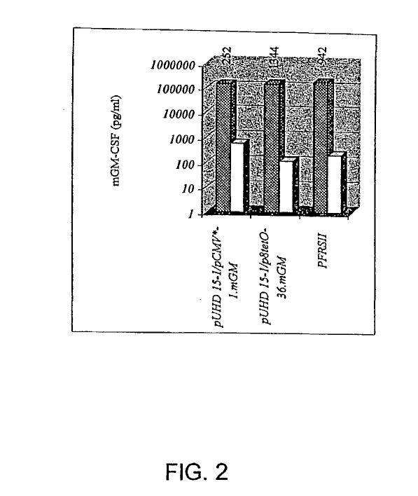

The results obtained from these studies indicate that the second generation tRP maintains its low basal expression and high regulation efficiency within the context of a positive feedback regulatable vector, as shown in FIG. 2. The PFRV II plasmid attains regulation efficiencies close to 900-fold in optimized experiments; a level several fold higher than the original co-transfected TRS. As far as we are aware, this is the highest regulation that has ever been reported from a single tetracycline-controlled plasmid. The single PFRV II plasmid is a simple and highly efficient regulatable vector that can be conveniently delivered via viral or non-viral means.

The data for FIG. 2 was generated as follows. HeLa cells in 6-well plates were co-transfected with 1 μg of PFRV II and 1 μg of pBR322, 1 μg of pCMV*⁻¹.mGM-CSF and 1 μg of pUHD 15-1, or 1 μg of p8tetO-36.mGM-CSF and 1 μg of pUHD 15-1, together with 0.5 μg of pCH110 as a control for assessing transfection efficiency. The final mGM-CSF value represents the average of 6 samples. The standard error of each average was consistently less than 10% of the final value. The experiment was repeated 2 times to establish a pattern.

### Example 4

Humanized Tetracycline-Regulated Transactivators: tetR-p65

As a chimeric regulatable system, the TRS may elicit an immune response that limits gene expression. Even though, gene expression and regulation have been observed for over 6 months, with no detection of anti-tTA antibody (Bohl et al., 1997), both tetR and VP16 domains are foreign and potentially immunogenic. To reduce this immunogenicity, we have replaced the VP16 domain of tTA with the C-terminal 286-551 and 313-551 amino acids of human NF-κB p65 protein and the efficiencies of these chimera were compared with that of tTA in co-transfection studies.

Both tetR-p65²⁸¹ and pCMV.tetR-p65³¹³ functioned efficiently as tetracycline-regulated transactivators, reaching regulation efficiencies comparable with that of tTA, as shown in Table 5, below. Similar observations have been reported in the context of the rapamycin-dependent transactivator of RRS (Rivera, V. M., Clackson, T., Natesan, S., Pollock, R., Amara J. F., Keenan, T., Magari, S. R., Phillips, T., Courage, N. L., Cerasoli, Jr., F., Holt, D. A., and Gilman, M. (1996). A humanized system for pharmacologic control of gene expression. Nature Medicine 2,1028-1032, incorporated herein by reference in its entirety) and RU486-dependent transactivator of PRS (Burcin, M. M., Schiedner, G., Kochanek, S., Tsai, S. Y., and O'Malley, B. W. (1999). Adenovirus-mediated regulable target gene expression in vivo. Proc. Natl. Acad. Sci. USA. 96,355-360, incorporated herein by reference in its entirety), and may reflect the close functional and structural relationship of p65 and VP16 (Schmitz, M. L., Dos Santos Silva, M. A., Altmann, H., Czisch, M., Holak, T. A., and Baeuerle, P. A. (1994). Structural and functional analysis of the NF-kappa B p65 C terminus. An acidic and modular transactivation domain with the potential to adopt an alpha-helical conformation. J. Biol. Chem. 269,25613-25620, incorporated herein by reference in its entirety). The new humanized tetracycline-regulated transactivator should reduce the potential immunogenicity of tTA, although it will not prevent an immune response against the tetR domain.

TABLE 5

| | | | Fold |
|---|---|---|---|
| Plasmid | −Dox | +Dox | Regulation |
| pUHD 15-1/ | 121320 | 136 | 892 |
| P8tetO-36.mGM | | | |
| pCMVtetR-p65²⁸⁶/ | 118230 | 120 | 985 |
| p8tetO-36.mGM | | | |
| pCMVtetR-p65³¹³/ | 133540 | 126 | 1060 |
| P8tetO-36.mGM | | | |

HeLa cells in 6-well plates were co-transfected with using 1 μg of pCMV.tetR-p65²⁸¹, pCMV.tetR-p65³¹³, or pUHD 15-1 and 1 μg of p8tetO-36.mGM-CSF, together with 0.5 μg of pCH110 as a control for assessing transfection efficiency. The final mGM-CSF value represents the average of 6 samples. The standard error of each average was consistently less than 10% of the final value. The experiment was repeated 2 times to establish a pattern.

### Example 5

Reducing the Basal Leakiness of the Minimal CMV Promoter in the Context of a Second Generation Tetracycline-Regulatable Promoter

Given the fact that the leakiness of the tetracycline regulatable promoter essentially reflects the basal expression of the minimal CMV promoter, various approaches are being explored to reduce this leakiness. The leakiness and maximal expression can be reduced either by using alternative minimal promoter, such as the promoter of thymidine kinase, or by reducing the minimal CMV promoter even further. The latter approach was explored by constructing a number of vectors that have deletions within the CMV promoter. The following sequence provides the first 500 bases of pUHD 10-3 (Gossen and Bujard, 1993). Bases 1 to 300 of the sequence include 7 tetO elements (bold, with central G underlined). The TATA box is situated between 341 to 347 (bold) and the first restriction site of the polylinker, EcoR1 (bold), is at 449 to 455. The minimal CMV IE promoter as it is know in the art is defined by bases 304 to 449. Targeted deletions in this sequence were made with the object to produce a promoter with lower maximal and basal expressions without compromising the fold regulation.

| | | |
|---|---|---|
| CTCGAGTTTA CCACTCCCTA TCAGTGATAG AGAAAAGTGA AAGTCGAGTT | 50 | (SEQ ID NO: 21) |
| TACCACTCCC TATCAGTGAT AGAGAAAAGT GAAAGTCGAC TTTACCACTC | 100 | |
| CCTATCAGTG ATAGAGAAAA GTGAAAGTCG AGTTTACCAC TCCCTATCAG | 150 | |
| TGATAGAGAA AAGTGAAAGT CGAGTTTACC ACTCCCTATC AGTGATAGAG | 200 | |
| AAAAGTGAAA GTCGAGTTTA CCACTCCCTA TCAGTGATAG AGAAAAGTGA | 250 | |
| AAGTCGAGTT TACCACTCCC TATCAGTGAT AGAGAAAAGT GAAAGTCGAG | 300 | |
| CTCGGTACCC GGGTCGAGTA GGCGTGTACG GTGGGA[GGCC TATATAAGCA | 350 | |
| GAGCTCGTTT AGTGAACCGT CAGATCGCCT GGA]GACGCCA TCCACGCTGT | 400 | |
| TTTGACCTCC ATAGAAGACA CCGGGACCGA TCCAGCCTCC GCGGCCCCGA | 450 | |
| ATTCGAGCTC GGTACCCGGG GATCCTCTAG AGGATCCAGA CATGATAAGA | 500 | |
| (Gossen and Bujard, 1992) | | |

A series of vectors have been constructed to produce specific but lower maximal expressions and reduced basal expressions. Plasmid p8tetO-16.mGM del R1-SacII has a deletion of sequence corresponding to bases 439 to 449 of pUHD 10-3. The plasmid p8tetO-16.mGM del R1-Bbs1 has a deletion of sequence corresponding to bases 414 to 449 of pUHD 10-3. Plasmid p8tetO-16.mGM del KpnI-StuI has a deletion of sequence corresponding to bases 304 to 336 of pUHD 10-3. p8tetO-16.TATAII.mGM. has a micro CMV promoter extending from 6 bases upstream of the TATA box to 36 bases downstream of TATA (bracketed above). As shown in Table 6, below, This vector displays significant reduction in the maximal expression of the promoter by 27-fold, with no detectable basal expression.

TABLE 6

| | | | Fold |
|---|---|---|---|
| Plasmid | −Dox | +Dox | Regulation |
| pUHD 15-1/ | 500000 | 125 | 4000 |
| p8tetO-36.mGM | | | |
| pUHD 15-1/ | 18000 | ND | |
| p8tetO-36-TATA.mGM | | | |

ND = Undetectable.

HeLa cells in 6-well plates were co-transfected with 1 μg of p8tetO-16-TATA.mGM or p8tetO-16.mGM and 1 μg pUHD 15-1 together with 0.5 μg of pCH110 as a control for assessing transfection efficiency. The final mGM-CSF value represents the average of 6 samples. The standard error of each average was consistently less than 10% of the final value.

The second generation tetracycline-regulated promoter functions well both in the context of a down-regulatory and up-regulatory systems, as shown in Table 7, below. As in the case of the down-regulatory system, the second generation tetracycline-regulated promoter also produces higher maximal expression and thus a higher degree of regulation in the context of the up-regulatory system (when using rtTA).

TABLE 7

| Plasmid | RtTA/Dox+ | RtTA/Dox− | Fold Regulation |
|---|---|---|---|
| pCMV*⁻².mGM | 198800 | 4158 | 47 |
| p2tetO-36.mGM | 318000 | 2389 | 133 |
| p4tetO-36.mGM | 302300 | 1329 | 227 |
| p6tetO-36.mGM | 335000 | 1411 | 237 |
| p8tetO-36.mGM | 372900 | 1443 | 258 |

HeLa cells in 6-well plates were co-transfected with 1 μg of p2tetO-36.mGM, p4tetO-36.mGM, p6tetO-36.mGM, or p8tetO-36.mGM and 1 μg pUHD 17-1 (Gossen et al., 1995) together with 0.5 μg of pCH110 as a control for assessing transfection efficiency. The final mGM-CSF value represents the average of 5 samples. The standard error of each average was consistently less than 10% of the final value.

We claim:

1. An isolated nucleic acid comprising a plurality of transactivator binding domains that are spaced such that when bound by transactivators, the transactivators are substantially rotationally aligned about the DNA helix.

2. The nucleic acid of claim 1, wherein the transactivator binding domains are tetO sequences.

3. The nucleic acid of claim 2, wherein the central nucleotides of adjacent tetO sequences are separated by 13-19, 23-29, 33-39 or 44-50 bases.

4. The nucleic acid of claim 2, wherein the central nucleotides of adjacent tetO sequences are separated by about 16, 26, 36 or 47 bases.

5. The nucleic acid of claim 2, wherein the central nucleotides of adjacent tetO sequences are separated by 16, 26, 36 or 47 bases.

6. The nucleic acid of claim 1, wherein the central nucleotides of adjacent binding domains are rotated with respect to each other by at least about 90° about the nucleic acid helix.

7. The nucleic acid of claim 1, wherein the central nucleotides of adjacent binding domains are rotated with respect to each other by about 180° about the nucleic acid helix.

8. The nucleic acid of claim 2, wherein at least one of the tetO sequences is a class B tetO sequence.

9. The nucleic acid of claim 8, wherein at least one of the tetO sequences is a class B2 tetO sequence.

10. The nucleic acid of claim 2, further comprising a promoter operably linked to the tetO sequence.

11. The nucleic acid of claim 10, wherein the promoter is one of a CMV promoter and a minimal CMV promoter.

12. The nucleic acid of claim 11, wherein the promoter consists of bases 337 to 383 of SEQ ID NO: 21.

13. The nucleic acid of claim 1, further comprising a viral packaging sequence for packaging the nucleic acid into a viral capsid.

14. The nucleic acid of claim 2, further comprising a promoter operably linked to the tetO sequences and an coding sequence operably linked to the promoter.

15. The nucleic acid of claim 14, further comprising a second promoter operably linked to the tetO sequences in an orientation on the nucleic acid opposite that of the first promoter and the second promoter is operably linked to a coding sequence of a transactivator or transrepressor for binding to at least one of the tetO sequences.

16. The nucleic acid of claim 2, comprising from 2 to 8 tetO sequences.

17. The nucleic acid of claim 16, comprising 8 tetO sequences.

18. The nucleic acid of claim 17, comprising a CMV promoter, or a derivative thereof, operably linked to the tetO sequences.

19. An isolated and purified nucleic acid, comprising:
a) from 2 to 8 class B tetO sequences, each tetO sequence having a central nucleotide, wherein said central nucleotide of each tetO sequence is separated from a central nucleotide of an adjacent tetO sequence on the nucleic acid by about 26, 36 or 47 nucleotides; and
b) a first promoter operably linked to the tetO sequences.

20. The nucleic acid of claim 19, wherein the first promoter is a CMV promoter or a derivative thereof.

21. The nucleic acid of claim 20, wherein the first promoter consists of nucleotides 337 to 383 of SEQ ID NO: 21.

22. The nucleic acid of claim 19, further comprising a second promoter operably linked to the tetO sequences and in an opposite orientation on the nucleic acid as the first promoter, wherein the second promoter is operably linked to a sequence encoding a tetracycline transactivator, a tetracycline transrepressor or a derivative thereof.

23. The nucleic acid of claim 22, wherein the first viral promoter and the second viral promoter are both CMV promoters.

24. The nucleic acid of claim 22, wherein the tetracycline transactivator is a fusion protein consisting of a substantially complete tetracycline repressor protein having a transactivation domain attached to its C-terminus.

25. The nucleic acid of claim 24, wherein the transactivation domain is selected from the group consisting of herpes simplex virus VP-16, Epstein-Barr Virus rta and human NF-κB p65 transactivators.

26. The nucleic acid of claim 19, comprising circular DNA.

27. The nucleic acid of claim 26, comprising a plasmid.

28. A modified cell comprising a nucleic acid comprising a gene having a regulatable promoter including a plurality of transactivator binding domains that are spaced such that when bound by transactivators, the transactivators are substantially rotationally aligned about the DNA helix.

29. The modified cell of claim 28, wherein the cell is a bacteria, yeast or mammalian cell.

30. The modified cell of claim 29, wherein the nucleic acid contains sequences for the propagation of the nucleic acid in the cell.

31. The modified cell of claim 29, wherein the nucleic acid contains sequences that permit integration of the nucleic acid in the cell genome.

32. The modified cell of claim 31, wherein the sequences that permit integration of the nucleic acid in the cell are Retrovirus or Adeno-associated virus sequences.

33. The modified cell of claim 28, wherein the nucleic acid further comprises a promoter operably linked to the tetO sequences and a coding sequence operably linked to the promoter.

34. A transgenic plant or plant part comprising a nucleic acid comprising a transgene having a regulatable promoter including a plurality of transactivator binding domains that are spaced such that when bound by transactivators, the transactivators are substantially rotationally aligned about the DNA helix.

35. A modified non-human animal comprising a nucleic acid comprising a transgene having a regulatable promoter including a plurality of transactivator binding domains that are spaced such that when bound by transactivators, the transactivators are substantially rotationally aligned about the DNA helix.

36. An isolated and purified nucleic acid comprising a CMV micro-promoter consisting essentially of bases 337 to 383 of SEQ ID NO: 21.

37. The nucleic acid of claim 36, further a plurality of transactivator binding domains operably linked to the micro-promoter that are spaced such that when bound by transactivators, the transactivators are substantially rotationally aligned about the DNA helix.

38. A method for expressing a gene in a cell comprising the steps of:
a) introducing a nucleic acid into the cell, the nucleic acid comprising a regulatable promoter comprising a plurality of transactivator binding domains that are spaced such that when bound by transactivators, the transactivators are substantially rotationally aligned about the DNA helix, wherein the plurality of transactivator binding domains are operably linked to a promoter operably linked to a coding sequence;
b) expressing a transactivator in the cell, such that expression of the coding sequence is activated under conditions where the transactivator binds the transactivator binding domain.

39. The method of claim 38, wherein the transactivator binding domains are tetO sequences and the transactivator is one of tTA and rtTA, which is encoded on and expressed from the same nucleic acid as the coding sequence.

40. The method of claim 38, wherein the nucleic acid is introduced into the cell by a method selected from the group consisting of viral transduction, liposome transfection, calcium phosphate precipitation, DEAE-dextran transformation, particle bombardment and microinjection.

41. A method for preparing an expressible gene comprising the step of ligating and thereby operably linking a nucleic acid comprising a coding sequence to a nucleic acid comprising a tetracycline-regulatable promoter comprising a plurality of tetO sequences operably linked to a promoter, wherein each tetO sequence has a central nucleotide and each central nucleotide of adjacent tetO sequences is separated by 23-29, 33-39 or 44-50 bases.

42. A method for controlling expression of a tetracycline-regulatable gene including the step of contacting a cell containing a nucleic including the tetracycline-regulatable gene with one of chlortetracycline, oxytetracycline, demethylchloro-tetracycline, methacycline, doxycycline and minocycline.

43. A method for modulating expression from a regulatable promoter including two or more transactivator binding DNA domains, comprising the step of modifying the distances between the domains.


SEQUENCE LISTING (21 sequences)

SEQ ID NO: 1 - DNA, 41 bp, Artificial Sequence
  p2tetO-21 insert
tccctatcagtgatagagagcgtccctatcagtgatagaga

SEQ ID NO: 2 - DNA, 45 bp, Artificial Sequence
  p2tetO-26 insert
tccctatcagtgatagagagtcgagctccctatcagtgatagaga

SEQ ID NO: 3 - DNA, 51 bp, Artificial Sequence
  p2tetO-31 insert
tccctatcagtgatagagagtcgacctcgagctccctatcagtgatagaga

SEQ ID NO: 4 - DNA, 56 bp, Artificial Sequence
  p2tetO-36 insert
tccctatcagtgatagagaagtcgacacgtctcgagctccctatcagtgatagaga

SEQ ID NO: 5 - DNA, 62 bp, Artificial Sequence
  p2tetO-42 insert
tccctatcagtgatagagaagtcgacacgtgtatacctcgagctccctatcagtgatagaga

SEQ ID NO: 6 - DNA, 66 bp, Artificial Sequence
  p2tetO-47 insert
tccctatcagtgatagagaagtcgacactgtagatctactctcgagctccctatcagtgatagaga

SEQ ID NO: 7 - DNA, 72 bp, Artificial Sequence
  p2tetO-52 insert
tccctatcagtgatagagaagtcgacactgtagatctgcactgactctcgagctccctatcagtgatagaga

SEQ ID NO: 8 - DNA, 77 bp, Artificial Sequence
  p2tetO-57 insert
tccctatcagtgatagagaagtcgacactgtagatctgcactgactgacagctcgagctccctatcagtgatagaga

SEQ ID NO: 9 - DNA, 31 bp, Artificial Sequence
  tetO forward oligonucleotide
tcgagctccctatcagtgatagagaaggtac

SEQ ID NO: 10 - DNA, 23 bp, Artificial Sequence
  tetO reverse oligonucleotide
cttctctatcactgatagggagc

SEQ ID NO: 11 - DNA, 11 bp, Artificial Sequence
  Random linker-18 forward oligonucleotide
cccggagacgt

SEQ ID NO: 12 - DNA, 11 bp, Artificial Sequence
  Random linker-18 reverse oligonuleotide
ctccggggtac

SEQ ID NO: 13 - DNA, 22 bp, Artificial Sequence
  Random linker 2 forward oligonucleotide
cccggaatcgagtcatcgacgt

SEQ ID NO: 14 - DNA, 22 bp, Artificial Sequence
  Random linker 2 reverse oligonucleotide
cgatgactcgattccggggtac

SEQ ID NO: 15 - DNA, 13 bp, Artificial Sequence
  Synthetic linker forward oligonucleotide
gtccctaaggtcg

SEQ ID NO: 16 - DNA, 14 bp, Artificial Sequence
  Synthetic linker reverse oligonucleotide
ggattccagcctag

SEQ ID NO: 17 - DNA, 12 bp, Artificial Sequence
  SGG linker forward oligonucleotide
taagtggcgggg

SEQ ID NO: 18 - DNA, 13 bp, Artificial Sequence
  SGG linker reverse oligonucleotide
gattccccgccac

SEQ ID NO: 19 - DNA, 13 bp, Artificial Sequence
  SGG linker-2 forward oligonucleotide
taagtggcgggat

SEQ ID NO: 20 - DNA, 14 bp, Artificial Sequence
  SGG linker-2 reverse oligonucleotide
catgatcccgccac

SEQ ID NO: 21 - DNA, 500 bp, Artificial Sequence
  tetO/minimal CMV tRP
ctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcggccccgaattcgagctcggtacccggggatcctctagaggatccagacatgataaga